(19) United States
(12) Patent Application Publication
(10) Pub. No.: US 2022/0386884 A1
(43) Pub. Date: Dec. 8, 2022

(54) MOTION GENERATING INTERVENTIONAL AID DEVICE, SYSTEM, AND METHOD

(71) Applicant: Ghassan S. Kassab, La Jolla, CA (US)

(72) Inventor: Ghassan S. Kassab, La Jolla, CA (US)

(21) Appl. No.: 17/835,928

(22) Filed: Jun. 8, 2022

Related U.S. Application Data
(60) Provisional application No. 63/208,446, filed on Jun. 8, 2021.

(51) Int. Cl. A61B 5/021; A61M 25/00

(52) U.S. Cl. CPC A61B 5/02133; A61M 25/0043; A61M 2025/0063

(57) ABSTRACT

Motion generating devices for imparting motion to interventional devices to aid in the use of such interventional devices, and methods of making and using the same. An exemplary motion generating device of the present disclosure comprises at least one motion source, such as a motor, voice coil actuator, or the like, and a mating surface configured to engage an interventional device, wherein the motion generating device can impart multimodal vibrational and/or rotational movement, both independently and/or simultaneously, to the interventional device thereby aiding in the advancement of the interventional device through the vasculature of a patient.

### PRIORITY & RELATED APPLICATIONS

The present application is related to, and claims the priority benefit of, U.S. Provisional Patent Application Ser. No. 63/208,446, filed Jun. 8, 2021. The entire contents of the aforementioned application is hereby incorporated by reference in its entirety into this disclosure.

### BACKGROUND

Guidewires, catheters and other percutaneous devices are used to navigate through tortuous curved vascular anatomy (e.g., arteries, veins, lymphatics, etc.). As these devices are advanced, there are many contact points between the device and the vasculature providing frictional forces that oppose the advancing forces on the device. The frictional forces are larger if the anatomy is more tortuous, curved, diseased (e.g., calcification, atherosclerosis, varicose, etc.), smaller in caliber, etc. To overcome the frictional contact forces with the vessel wall, the clinician will shake the wire to generate longitudinal waves or vibrations that disrupt the contact points of friction. This can allow slight repositioning of the wire and additional attempts at re-advancement. Some clinicians will also advance the guidewire while shaking which requires technical coordination and experience. This shaking or flicking of the device causes significant reduction in friction thereby, enabling easier advancement of the device.

The shaking process is subjective and requires energy which can be draining after long procedures. Excessive shaking or overly forceful shaking can cause unintended consequences such as perforation of vasculature. Thus, tempering the degree and amount of shaking is a skill that must be learned through thorough training, over the course of time, and after multiple procedures. Furthermore, as each patient's vasculature is different, and the frictional forces involved can vary due to the diseases, tortuous geometry and uniqueness of each patient vasculature, the degree of shaking required when advancement of the guidewire is stopped may be different at each instance, further complicating procedures involving these devices.

Therefore, there is a need for a tool that can accomplish the same in a more reproducible and systematic approach and that does not require experience especially for the less experienced operators.

### BRIEF SUMMARY

In an exemplary embodiment of a motion generating device of the present disclosure, the motion generating device comprises a mating surface and at least one motion source.

In an alternate embodiment of a motion generating device of the present disclosure, the motion generating device comprises a housing, at least one motion source disposed within the housing, a power source powering the at least one motion source, and a mating surface on the housing.

In an alternate embodiment, the motion generating device comprises at least one motion source, a housing comprising a mating surface, wherein the mating surface is on the housing and is part of the housing. In an alternate embodiment, the motion generating device comprises at least one motion source, a housing and a mating surface wherein the mating surface is a separate element disposed on the housing.

In an exemplary embodiment of a motion generating device of the present disclosure, the motion generating device comprises at least one motion source, a mating surface, and a housing having a clam-shell shape, wherein the housing comprises a housing top portion comprising a top portion of a mating surface and a housing bottom portion comprising a bottom portion of a mating surface, wherein the top portions of the housing and the mating surface and the bottom portions of the housing and the mating surface are configured to fold over an interventional device in a clam shell fashion.

In an alternate embodiment, the power source may be disposed within the housing. In alternate embodiments, the at least one motion source or the power source is disposed external of the housing. The power source and the at least one motion source are operably connected such that the at least one motion source is supplied energy from the power source.

The interventional device may comprise a guidewire, catheter, deployment handle, or any other percutaneous device designed to traverse the vasculature of a patient.

The at least one motion source may comprise a voice coil actuator, a small electric motor with an unbalanced mass on the driveshaft, or a combination of both to generate multimodal vibrational and rotational movement, both independently of one another and simultaneously.

The motion generating device may further comprise at least one switch operable to toggle the power source and/or the at least one motion source on and off. The motion generating device may also comprise at least one switch operable to vary the characteristics of the vibrations and/or rotations/torque generated such as the amplitude, frequency and/or rotational speed produced.

In an alternate embodiment, the motion generating device may be one piece with the interventional device. That is, the motion generating device may be pre-formed or otherwise manufactured as a single piece with the interventional device.

In an exemplary embodiment of a method for using a motion generating device of the present disclosure, the method comprises the steps of positioning the motion generating device on an interventional device and activating the motion generating device.

In an alternate embodiment of a method for using a motion generating device of the present disclosure, the method comprises the steps of positioning the motion generating device on an interventional device, attaching the motion generating device to the interventional device and activating the motion generating device.

In a further alternate embodiment, the motion generating device is preferably positioned on the proximal end of the interventional device, the proximal end being the end furthest from the patient.

Activating the motion generating device may further comprise at least one or more of the steps of connecting the power source to the motion generating device, activating at least one switch operable to toggle the power source on, activating at least one switch operable to turn the at least one motion source on.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1 shows a schematic representation of a motion generating device, according to an exemplary embodiment of the present disclosure;

FIG. 2 shows a schematic representation of a motion generating device, according to an alternate embodiment of the present disclosure;

FIG. 3 shows a schematic representation of a motion generating device, according to an alternate embodiment of the present disclosure;

FIG. 4 shows a side view of an exemplary embodiment of a motion generating device of the present disclosure in an open position; and

FIG. 5 shows a side view of an exemplary embodiment of a motion generating device of the present disclosure in a closed position.

An overview of the features, functions and/or configurations of the components depicted in the various figures will now be presented. It should be appreciated that not all of the features of the components of the figures are necessarily described. Some of these non-discussed features, such as various couplers, etc., as well as discussed features are inherent from the figures themselves. Other non-discussed features may be inherent m component geometry and/or configuration.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

It is within the scope of the invention to solve the problems present in the prior art, such as overcoming friction caused by advancing a lengthy device through a curved lumen, in any technical field. For example, the device may be used in colonoscopy, bronchoscopy, or any other endoscopic or minimally invasive procedure. The device could also be adapted and scaled for use in nonmedical settings such as plumbing or other industrial probe advancement applications. The embodiments within are illustrative of the invention and intended to be non-limiting. Similarly, the cardiovascular examples are given only for context.

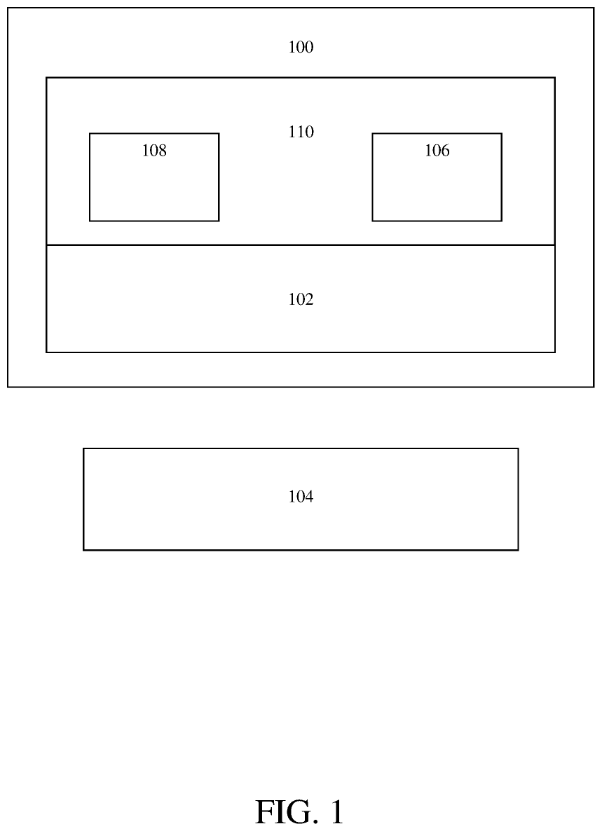

An exemplary device for a motion generating device 100 of the present disclosure is shown in FIG. 1. As shown in FIG. 1, the motion generating device 100 comprises at least one motion source 106 and a mating surface 102. The at least one motion source 106 and the mating surface 102 are operably connected such that movement, e.g., vibrations, rotations, etc., from the at least one motion source 106 can be transferred to an interventional device 104 attached to the mating surface 102. The mating surface 102 is configured to mate with an interventional device 104.

As seen in FIG. 1, the at least one motion source 106 may be disposed within a housing 110, and the mating surface 102 is disposed on the housing 110. In use, the motion generating device 100 is in contact with an interventional device 104 via the mating surface 102. Thus, when the at least one motion source 106 produces movement, e.g., vibrations, rotations, etc., the movement will travel from the at least one motion source 106, to the housing, to the mating surface 102 and finally to the interventional device 104.

The vibrations and/or rotations may travel along the length of the interventional device 104. For example, if the motion generating device 100 is attached to the proximal end of the interventional device 104, the vibrations and/or rotations may travel from the proximal end of the interventional device 104 to the distal end. In this manner, the motion generating device 100 replaces the physical movement of the interventional device operator. Then, the operator may focus his or her energy on advancing the interventional device 104 as the motions are being generated.

The mating surface 102 is configured to mate with the interventional device 104. The mating surface 102 may be a part of the housing 110 and on the exterior of the housing 110, or may be a separate element and on the housing 110. The mating surface 102 may comprise a deformable material such that when the housing 110 is placed over the interventional device 104 the material will take the general shape of the interventional device 104. In this embodiment, the deformable material will be softer than the interventional device 104. In another embodiment, the mating surface 102 may comprise a frictional material that prevents movement between the mating surface 102 and the interventional device 104. The mating surface 102 may also be pre-formed to take the general shape of the interventional device 104.

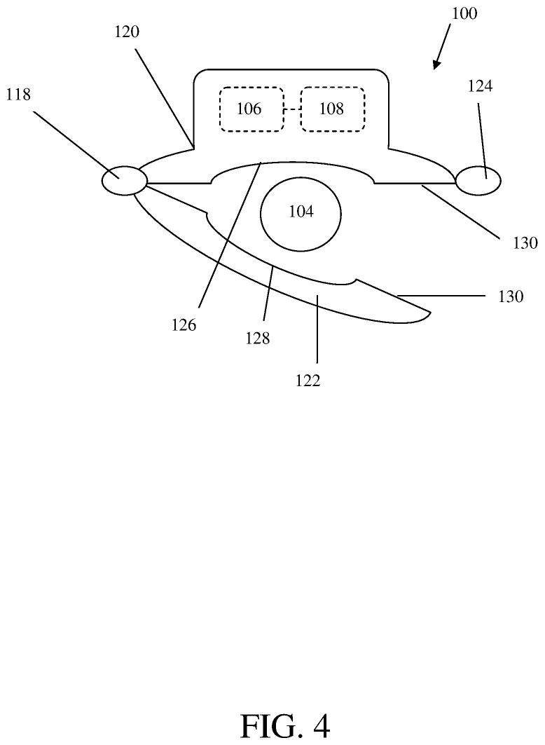

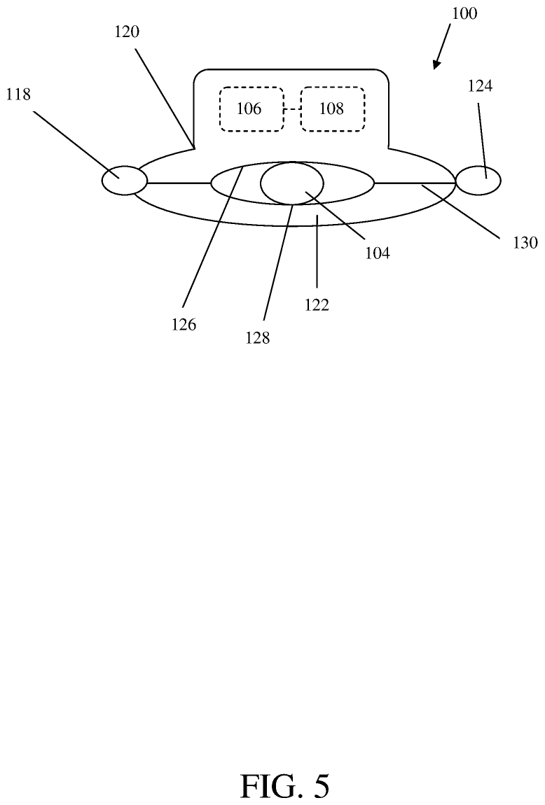

In another embodiment, the motion generating device 100 has a foldable “clam-shell” shaped housing 110 that aids in attachment to the interventional device 104. A side view of this embodiment is illustrated in FIGS. 4-5. The housing 110 may comprise a top portion 120, and a bottom portion 122 connected by a hinge 118. In this embodiment, the top portion 120 and the bottom portion 122 of the housing 110 each also comprise the mating surface 102, thereby creating a top portion of the mating surface 126 and a bottom portion of the mating surface 128. The top portion of the mating surface 126 is on the top portion of the housing 120 and the bottom portion of the mating surface 128 is on the bottom portion of the housing 122. The housing 110 may also further comprise a securing mechanism 124.

The hinge 118 allows the housing 110 to be in a first position and a second position. In the first position as shown in FIG. 4, the housing 110 is in an open position wherein the top portion 126 and the bottom portion 128 of the mating surfaces 102 are separated at an end 130 so that the top portion 126 and the bottom portion 128 of the mating surfaces 102 are separated along at least most of their lengths. In this position the interventional device 104 may freely move in and out of the motion generating mechanism.

As shown in FIG. 5, in the second position, the housing 110 is in a closed position. In this position the top portion 126 and bottom portion 128 of the mating surface 102 are lying together along at least a portion of their length, including at said end 130. The interventional device 104 is located between the top portion 126 and the bottom portion 128 of the mating surface 102 and secured by the pressure of the top and bottom portions 126, 128 of the mating surface 102. When the interventional device 104 is secured between the top portion and the bottom portion of the housing 120, 122 and mating surface 126, 128, the movement of the at least one motion source 106 can be transferred to the interventional device 104 as described above. The securing mechanism 124 can be used to further hold the housing 110 in the second, closed position. The securing mechanism 124 may increase the pressure within the top and bottom surfaces of the mating surface 126, 128 or simply maintain the housing 110 in a closed position.

It is within the scope of this disclosure that the mating surface 102 may have one or more of the characteristics described in the preceding embodiments. For example, the mating surface 102 may have a top portion and a bottom portion 126, 128, and may also be comprised of a deformable material. As another example, the mating surface 102 may be pre-formed to fit an interventional device 104 and may also comprise a frictional material. It is also within the scope of this disclosure that the interventional device 104 may be secured in the housing 110 by being enclosed by a mating surface top portion 126 and mating surface bottom portion 128. It is also within the scope of this disclosure that the motion generating device 100 may be coupled to the interventional device 104 via compression collet, or similar means.

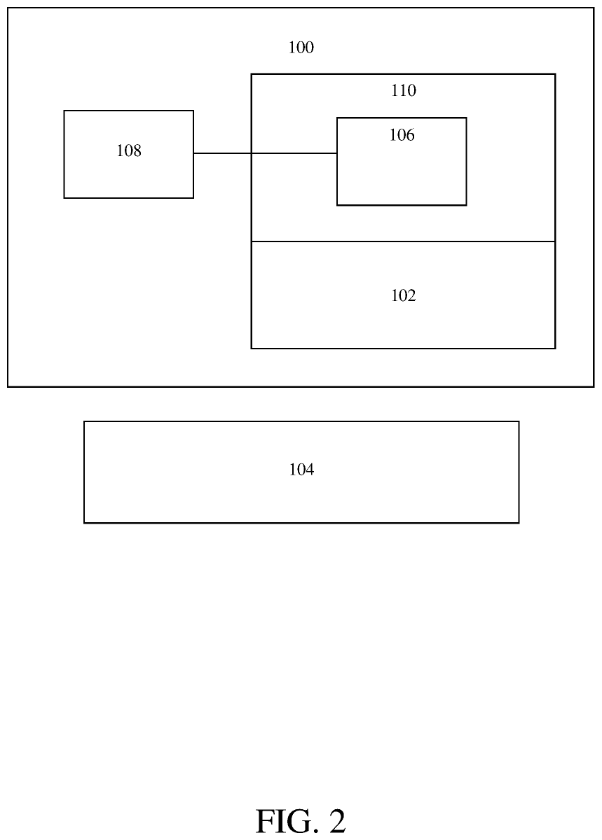

The motion generating device may further comprise a power source 108. The power source 108 may be located inside the housing, as in FIG. 1, or it may be located outside the housing 110 as in FIG. 2. In one embodiment, the power source 108 comprises a battery.

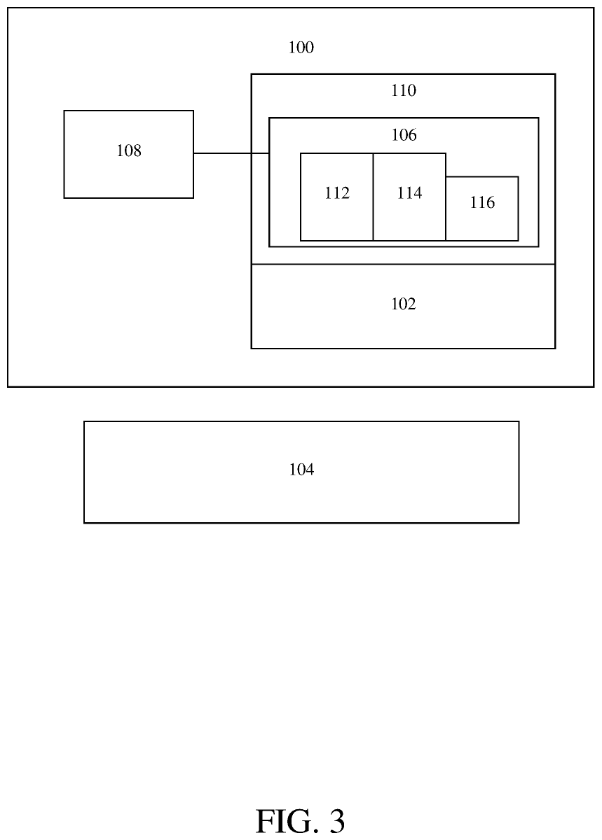

As shown in FIG. 3, the at least one motion source 106 may comprise a motor 112. The motor 112 may comprise a driveshaft 114 and an unbalanced mass 116 on the driveshaft 114 for generating vibration. The motor 112 may also be configured to provide rotational motion/torque to the interventional device 104. Alternatively, the at least one motion source 106 may comprise multiple motors, a voice coil actuator, or a combination of a motor(s) and a voice coil actuator.

The motion generating device 100 thus functions to provide vibrations and/or rotational motion and advance the interventional device 104 as the vibrations/rotational motion are transmitted along the interventional device 104. The motion generating device 100 may thus facilitate multimodal vibrational and rotational movement, both independent of one another and simultaneously.

The motion generating device 100 is also capable of generating different wavelengths that are transmitted along the length of the interventional device 104. For example, the motion generating device may comprise switches, dials, toggles or similar devices which can turn on and off the power source 108 and motion source 106. The switches may also function to alter the wavelength characteristics of the vibrations generated. In one embodiment, wavelength is altered by modifying the speed of the motor 112 via a multiposition switch. In another exemplary embodiment, the energy of the power source 108 is varied via a multiposition switch.

An exemplary method of using the interventional device comprises the steps of attaching the motion generating device to the interventional device, the motion generating device being configured to generate multimodal vibrational and rotational movement, both independent of one another and simultaneously, and activating the motion generating device to impart the multimodal vibrational and rotational movement to the interventional device, both independent of one another and simultaneously.

In an alternate embodiment, the method further comprises the step of attaching the motion generating device to the proximal end of the interventional device.

An alternate embodiment of a method of using the interventional device comprises the steps of inserting the interventional device into a patient, attaching the motion generating device to the interventional device, activating the motion generating device.

In an alternate embodiment, a method of using the interventional device further comprises the steps of advancing the interventional device while the motion generating device is activated.

In an alternate embodiment, a method of using the interventional device further comprises the steps of adjusting the at least one wavelength characteristic of the generated vibrations.

In an alternate embodiment, a method of using the interventional device further comprises the steps of advancing the interventional device while adjusting at least one wavelength characteristic of the generated vibrations.

In an alternate embodiment a method of using the interventional device further comprises the steps of adjusting at least one wavelength characteristic of the generated vibrations a first time after advancing the interventional device, and adjusting at least one wavelength characteristic of the generated vibrations a second time.

While various embodiments of devices for a motion generating device and methods for using a motion generating device to advance an interventional device and the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

1. A motion generating device comprising:
at least one motion source; and
a mating surface.

2. The motion generating device of claim 1 further comprising a housing, wherein the mating surface is disposed on the housing.

3. The motion generating device of claim 2 wherein the mating surface is a portion of an exterior of the housing.

4. The motion generating device of claim 1 wherein the mating surface comprises a mating surface top portion and a mating surface bottom portion.

5. The motion generating device of claim 4, wherein the motion generating device has an open position, and a closed position; and
wherein the mating surface top portion and the mating surface bottom portion are separate from each other along their lengths in the open position; and
wherein the mating surface top portion and the mating surface bottom portion are in contact with each other along at least a portion of their lengths in the closed position.

6. The motion generating device of claim 1, wherein the at least one motion source comprises a first motion source and a second motion source.

7. The motion generating device of claim 6, wherein the first motion source is configured to generate vibrational motion, and the second motion source is configured to generate rotational motion.

8. A motion generating device comprising:
a housing comprising a housing top portion and a housing bottom portion, a mating surface on the housing, and at least one motion source disposed within the housing;
the mating surface comprises a mating surface top portion and a mating surface bottom portion wherein the mating surface top portion is disposed on the housing top portion and the mating surface bottom portion is disposed on the housing bottom portion;
the motion generating device comprises an open position and a closed position; wherein the mating surface top portion and the mating surface bottom portion are separated at an edge in the open position; and
wherein the mating surface top portion and the mating surface bottom portion are in contact along at least a portion of their lengths in the closed position.

8. The motion generating device of claim 7 further comprising:
a hinge wherein the housing top portion and the housing bottom portion are connected by the hinge.

9. The motion generating device of claim 7 further comprising a securing mechanism configured to hold the housing top portion and the housing bottom portion together.

10. The motion generating device of claim 7, wherein the at least one motion source comprises a first motion source and a second motion source.

11. The motion generating device of claim 10, wherein the first motion source is configured to generate vibrational motion, and the second motion source is configured to generate rotational motion.

12. A method for using a motion generating device to advance an interventional device the method comprising the steps of:
inserting an interventional device into a patient;
attaching a motion generating device to the interventional device;
activating the motion generating device; and
advancing the motion generating device.

13. The method for using a motion generating device to advance an interventional device of claim 12 further comprising the steps of:
adjusting characteristics of the motions generated by the motion generating device; and
advancing the interventional device after adjusting the characteristics of the motions generated by the motion generating device.
